(19) United States
(12) Patent Application Publication
(10) Pub. No.: US 2025/0186638 A1
(43) Pub. Date: Jun. 12, 2025

(54) Method and Technique for the Focusing of UVC Light Energy to a Focused Energy Beam

(71) Applicants: Ronald Scott Frazier, Boulder City, NV (US); Nicholas A. Connelly, Ballston Spa, NY (US); William E. Ray, Dededo, GU (US); Hiroaki Yokoi, Torrance, CA (US)

(72) Inventors: Ronald Scott Frazier, Boulder City, NV (US); Nicholas A. Connelly, Ballston Spa, NY (US); William E. Ray, Dededo, GU (US); Hiroaki Yokoi, Torrance, CA (US)

(21) Appl. No.: 18/920,945

(22) Filed: Oct. 20, 2024

Related U.S. Application Data
(60) Provisional application No. 63/545,114, filed on Oct. 20, 2023.

(51) Int. Cl. A61L 2/10; G02B 1/00; G02B 13/00; G02B 13/14

(52) U.S. Cl. CPC A61L 2/10; G02B 1/00; G02B 13/0095; G02B 13/143

(57) ABSTRACT

An array that focuses UVC light energy from a UVC light source into a beam and thereby reduces the degradation of UVC light energy at a set distance. Singular or plural optics, or lens assemblies forming the array can each use a UVC beam total internal reflection (TIR) lens which transmits a UVC beam which allows an extended distance greater that the fall off rate of the standard UVC energy source.

This application claims the benefit of US Provisional Application Ser. No. 63/545,114, filed Oct. 20, 2023.

### BACKGROUND

It has been noted that UVC light energy produced by UVC diodes and UVC tube/lamps are radiated in a wide angle (degrees). UVC diodes typically have 120 degrees to 150 degrees radiation pattern, UVC tubes can radiate up to a 360-degree radiation pattern and UVC Lamps can radiate in a conical pattern at up to 150 degrees.

It is also noted that the light energy degradation is expressed by an inverse-square law. This degradation is known as fall-off.

It is common practice to use reflectors or other light mirrors to direct the rays of light to the desired target area. These methods are inefficient and rely on the reflecting of light rays off a reflective material to achieve a desired light direction.

U.S. Pat. No. 8,021,608 and 8,318,089; and US Published Patent Application 2018/0343847 disclose UVC sterilizing devices and are herein incorporated by reference.

The present inventors have recognized that it would be desirable to direct UVC radiation to a desired position with minimal degradation of UVC light energy.

### SUMMARY

The exemplary embodiments of the invention focus UVC light energy from a UVC light source into a beam and thereby reduces the degradation of UVC light energy at a set distance.

Singular or plural optics or lens assemblies can each use a Diffractive Optical Element (“DOE”) with a beam width of UVC energy with acceptable transmission through the DOE, which transmits a UVC beam through a relay lens which thereby allows an extended distance greater that the fall off rate of the standard UVC energy source. A lens assembly can include a series of spacers and lenses that allow the manipulation of a wide angle UVC light source for the purpose of focusing the light to a desired beam shape, i.e., a thin line or bar, or a pin point. Each of the optics or lens assemblies may have one or more spacers having a specific width to add to the total beam shaping of the lenses.

Alternatively, the DOE and the relay lens combination can be replaced by a UVC Beam TIR lens.

The invention includes a UVC apparatus for focusing a UVC light source into a beam having increased energy at a set distance.

The apparatus can comprise a Diffractive Optical Element (“DOE”) transmitting a beam width of UVC energy which is then transmitted through a relay lens which thereby allows an extended distance greater that the fall off rate of the standard UVC energy source.

The UVC apparatus can include a spacer set at a specific width to add to the total lens effect.

Alternatively, an alternate UVC apparatus, particularly useful as a beam water sterilizer, utilizes a Total Internal Reflection (TIR) UVC lens to replace the combination of DOE and relay lenses. This TIR UVC lens is placed into a Lens Mount. This lens mount is then coupled to a UVC LED circuit board. The board then has a heat sink attached by screws to dissipate heat away from the circuit board. The heat sink has mounting studs to hold the LED driver circuit board. The assembly is then placed into a cylinder casing which has a heat reduction fan mounted onto an end.

To utilize as much light as possible, a large angle is collected from the light source. With a solely refractive lens or a solely reflective lens, the size of the optical part becomes much too large for efficient collection of the light. For this case, both refractive and reflective properties are considered in the optical design.

The front portion of the lens is refractive, while the side surfaces are reflective due to total internal reflection. Such a lens is the TIR lens. Although the TIR lens uses both refractive and reflective properties of the lens, it is typically called a TIR lens since a large part of the optical design relies on total internal reflection.

Examples of TIR lenses are shown in US Published Patent Applications 2023/0129349; 2017/0205032 and 2012/0140462, herein incorporated by reference.

Present water sterilization devices utilize UVC gas tubes. UVC tubes utilize low vapor pressure mercury to emit UV light at 253.7 nm. The alternate UVC apparatus uses a TIR lens and assembly to produce a UVC beam which is projected into the oncoming water. This unit is positioned onto a similar chamber as the UVC mercury tube. However, instead of being placed into the tube, this unit is mounted at the head of the chamber. Having the water run against the UVC light beam causes the sterilization of bacteria due to the residence time within the UVC energy.

For water sterilizing, the assembly is threaded at the lens end for easy attachment to existing piping. This device can be scaled to larger sizes to include more lenses to accommodate larger water sterilization systems.

The UVC apparatus is not limited to water sterilizing, but can be adapted for other sterilizing functions.

Numerous other advantages and features of the present invention will be become readily apparent from the following detailed description of the invention and the embodiments thereof, and from the accompanying drawings.

### BRIEF DESCRIPTION OF THE DRAWING

In the following detailed portion of the present description, the teachings of the present application will be explained in more detail with reference to the example embodiment shown in the drawings, in which:

FIG. 1 is a schematic exploded side view of an exemplary embodiment UVC lens assembly.

FIG. 2 is a schematic exploded side view of the UVC lens assembly of FIG. 1 mounted to a UVC LED diode according to an exemplary embodiment.

FIG. 3 is a schematic side view of the UVC lens assembly mounted to a UVC LED diode as shown in FIG. 2 in a lens assembly configuration of a four-assembly array according to another exemplary embodiment, with a top lens assembly shown in exploded fashion and the remaining three lens assemblies shown assembled.

FIG. 4 is a schematic side view of the UVC lens assembly mounted to a UVC LED diode as shown in FIG. 2 in a lens assembly configuration of a five-assembly array to produce a beam multiplier according to another exemplary embodiment.

FIG. 5 is a schematic plan view of the UVC lens assembly mounted to a UVC LED diode as shown in FIG. 2 in a honeycomb configuration to produce a beam multiplier according to another exemplary embodiment.

FIG. 6 is a schematic plan view of the UVC lens assembly mounted to a UVC LED diode as shown in FIG. 2 arranged in a straight-line configuration to produce an energy beam at a specified width and distance according to another exemplary embodiment.

FIG. 7 is a schematic side view of the UVC lens assembly mounted to a UVC LED diode as shown in FIG. 2 arranged in a straight-line configuration to produce an energy beam at a specified width and at a specified distance according to another exemplary embodiment.

FIG. 8 is a schematic end view of a four-sided device incorporating the UVC lens assembly mounted to a UVC LED diode as shown in FIG. 2 according to another exemplary embodiment.

FIG. 8A is a schematic enlarged end view taken from FIG. 8.

FIG. 9 is a schematic sectional view taken generally through plane 9-9 in FIG. 8.

FIG. 10 is a schematic side view of the UVC lens assembly mounted to a UVC LED diode as shown in FIG. 2 to produce a beam multiplier according to another) exemplary embodiment.

FIG. 11 is a schematic exploded side view of the UVC lens assembly mounted to a UVC LED diode as shown in FIG. 2 in an array to change the output energy of a UVC tube light according to another exemplary embodiment.

FIG. 12 is a schematic side view of another embodiment UVC lens assembly mounted adjacent to a UVC LED diode array.

FIG. 13 is a top perspective view of another embodiment UVC lens assembly mounted adjacent to a UVC LED diode array.

FIG. 14 is a top perspective view of another embodiment UVC lens assembly mounted adjacent to a UVC LED diode array.

FIG. 15 is an exploded perspective side view of an alternate embodiment utilizing a TIR UVC lens.

FIG. 16 is sectional view taken through plane 16-16 of FIG. 17, of a TIR lens in a lens holder of an alternate embodiment.

FIG. 17 is right side view of the alternate embodiment shown in FIG. 16.

FIG. 18 is sectional view taken through plane 18-18 of FIG. 19, of a TIR lens of the altemate embodiment of FIG. 16.

FIG. 19 is right side view of the TIR lens shown in FIG. 18.

FIG. 20 is a schematic sectional view of a TIR lens showing the projected light beams out of the lens.

### DETAILED DESCRIPTION

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

This application incorporates by reference U.S. Provisional Application Ser. No. 63/545,114, filed Oct. 20, 2023 and continuation-in-part U.S. application Ser. No. 17/362,663, filed. Jun. 29, 2021 in their entireties.

In the following detailed description, the DOE and relay lens are produced from a doped Si0₂ wafer growth or glass mold using a wafer comprised of SiO₂ and other impurities/dopants. Utilizing a designed mask for the lens shape, the wafer is cut or etched, and the lens produced. A second process uses a fabricated mold to accomplish the making of the lens out of the doped SiO₂ material. The second method is also accomplished by using UV transmitting filters which contain compositions using other glass and/or silicon type of material.

It is noted that polymers that can pass UVC energy can be used instead of, or in combination with, SiO₂ materials in the production of the invention.

Doped SiO₂ material is designed to pass UVC at frequencies in the UVC range with none or some energy loss at <70% percentage.

This design has several optical elements, spacers and a mounting fixture. The first element is for the focusing and shaping of light from a given UVC emitter with narrow light output of any desired shape. This element is called the “DOE.”

A second optical element is called the “relay lens.” The relay lens projects the energy at a controlled shape, either line or point at a specified distance.

The spacers, when used, between the UVC emitter, DOE and the Relay lens are used together to align and position the element to optimize the light energy.

This combination of spacers and optical elements allows for greater output energy at a given distance.

Finally, the groups of components are fastened, utilizing a framed mount, to the Printed Circuit Board (PCB) which a UVC SMD (surface mounted device) is mounted. A second application is to the UVC tube, whereas UVC Beam energy lenses can be applied in an array to accomplish a focalized beam pattern at a specified distance. It is noted that the application of this invention can be applied to UVC tube lights and UVC bulbs.

It is also noted that the invention can be applied to present UVC laser technology.

Light degrades over distance which follows the inverse square law 1/R². R=distance from UVC source to target location. This light falls off exponentially and creates problems when trying to design a device using UVC technology. According to embodiments of the invention, a higher output can be achieved at a given distance greater than the original source could previously obtain.

For example: for a 100 mW UVC LED, the total light output from the LED will be spread across a 120-150-degree angle. The light is dispersed very broadly and ultimately diluting its effectiveness at greater distances. For using UVC light at greater distances, it may be necessary to add more light sources (UVC LEDs or Lamps) to accommodate the loss in power due to the inverse square law.

But now, with this invention, light energy can be consolidated and project more photons in a desired beam size and shape.

This focusing increases the total power at the distance that is targeted, allowing more of the UVC light to reach the target, without having to add more light sources.

An approximate four order of magnitude increase in power output is calculated at a target location at a given distance from source, compared to having a standard light source reaching that same target location.

The focusing technology can be applied in different beam paths and shapes. This allows for unique applications and provides an easy solution for hard-to-reach areas. The shaping of the beam is advantageous based on user application, with the flexibility of changing beam widths and shapes, the technology can be used for all UVC light sources between 200 nm-280 nm wavelengths.

The exemplary embodiments described herein could drastically increase cost savings, decrease power consumption, and decrease UVC irradiation time, all with using less of the UVC source being applied. The exemplary embodiments provide enhanced safety, in that by focusing UVC radiation, UVC radiation in areas that are not of interest is reduced.

As described below an alternate embodiment replaces the DOE and relay lenses with a TIR UVC lens.

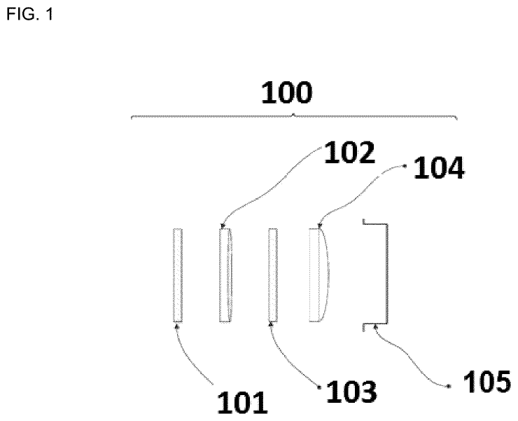

FIG. 1 depicts an exemplary embodiment of UVC lens assembly 100. The lens assembly 100 may includes a spacer 101; a DOE lens 102; a second spacer 103, and a relay lens 104, in order. Spacers 101 and 103 may or may not be required for assembly. The relay lens 104 includes a mount 105 for the lens assembly 100 to attach to a UVC LED board. The spacers 101, 103 can be hollow cylinders allowing UVC light to pass through the spacers.

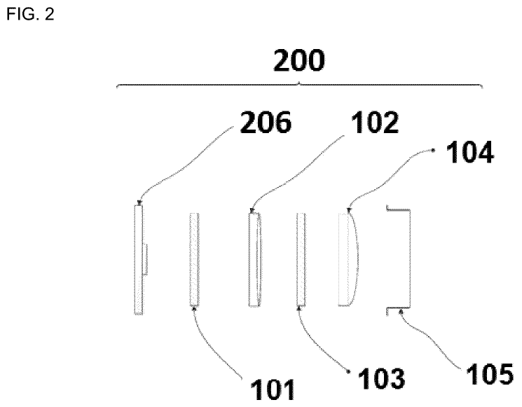

FIG. 2 depicts an exemplary embodiment of UVC emitter-lens assembly 200. The assembly 200 includes the assembly 100 from FIG. 1 mounted to a UVC emitter assembly 206. The assembly 206 includes a UVC LED circuit board containing a UVC element/UVC LED.

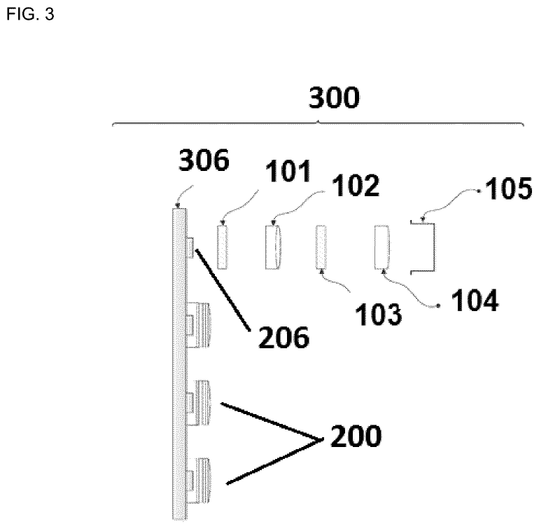

FIG. 3 depicts an emitter-lens array 300 having a plurality (such as four) of UVC emitter-lens assemblies 200. The emitter-lens assemblies 200 are mounted onto a UVC LED circuit board assembly 306.

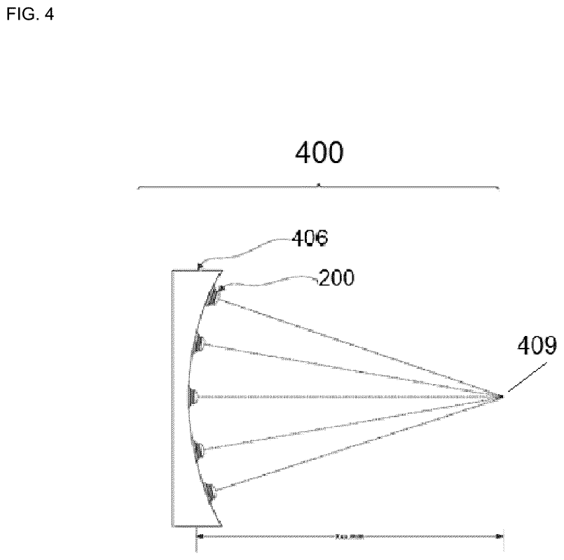

FIG. 4 depicts an emitter-lens array 400 having a plurality (such as five) of UVC emitter-lens assemblies 200. The emitter-lens assemblies are mounted onto a shaped circuit board or heat sink 406, which can be shaped in an arc or in a parabolic shape, directing the emitter-lens assemblies 200 to increase UVC energy at a specific focal point 409.

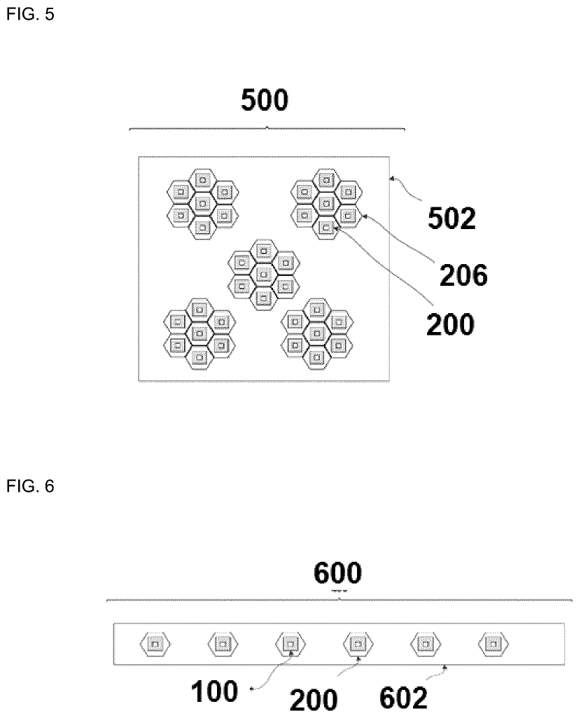

FIG. 5 depicts an emitter-lens array 500 having a plurality of UVC emitter-lens assemblies 200 in a honeycomb shaped pattern on a circuit board or heat sink 502. Each UVC emitter-lens assemblies 200 has a hexagon shape. The circuit board 502 carries UVC emitter-lens assemblies 200 to increase UVC energy at a specific beam width pattern or specific beam focal point.

FIG. 6 depicts an emitter-lens array 600 having a plurality (such as six) of UVC emitter-lens assemblies 200 arranged in a linear pattern on a circuit board or heat sink 602. Each UVC emitter-lens assemblies 200 has a hexagon shape. The UVC emitter-lens assemblies 200 increase UVC energy at a specific beam width.

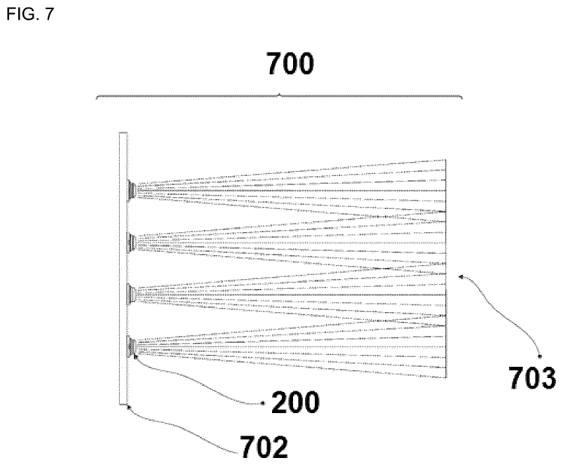

FIG. 7 depicts an emitter-lens array 700 having a plurality (such as four) of UVC emitter-lens assemblies 200 arranged in a linear pattern on a circuit board or heat sink 702. The UVC emitter-lens assemblies 200 increase UVC energy beam at a specific focus width and distance to a focus beam point 703.

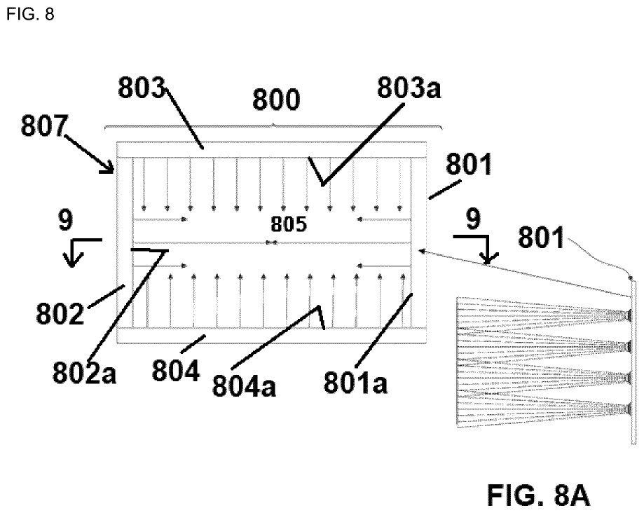

FIGS. 8 and 8A depict an exemplary embodiment of an emitter-lens array 800 having a plurality of UVC emitter-lens assemblies 200. The emitter-lens array 800 is incorporated onto UVC LED mounted circuit boards or heat sinks 801, 802, 803, 804 each carrying plural UVC emitter-lens assemblies 200 in an array 801a, 802a, 803a, 804a, and arranged in a rectangle and extending into the page and forming a four-sided UVC sterilization device 807 with an interior volume 805. Each array 801a, 802a, 803a, 804a incorporates UVC emitter-lens assemblies 200 directing UVC rays into the volume 805 to increase UVC energy at a specific focus width and distance. In FIG. 8, arrows are shown representing the direction of UVC light rays emitted from each UVC emitter-lens assembly 200. Each board or heat sink 801, 802, 803, 804 can be the assembly 700 depicted in FIG. 7.

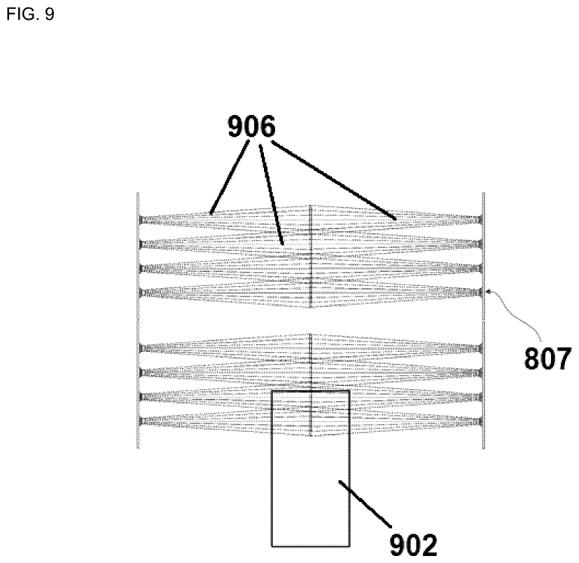

FIG. 9 Is a sectional view of the device 807 with an object 902 passing through the UVC energy beams 906. The UVC emitter-lens assemblies' 200 increases UVC energy at a specific focus width and distance to allow the UVC energy to fully cover the object at a distance greater that regular UVC LED components can presently accomplish.

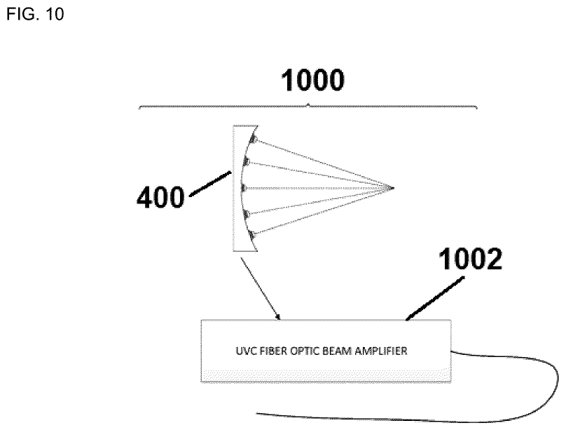

FIG. 10 depict an exemplary embodiment of an assembly 1000 including the emitter-lens array 400 from FIG. 4 having a plurality of UVC emitter-lens assemblies 200 incorporated into a UVC Fiber Optics Amplifier device 1002. The assembly 400 is enclosed within the UVC Fiber Optics Amplifier device 1002. UVC energy is focused to a single point leaving the UVC Fiber Optics Amplifier device which enables UVC fiber optic application. By utilizing the focus beam energy assembly, we can thereby increase the necessary UVC energy output into a fiber optic cable.

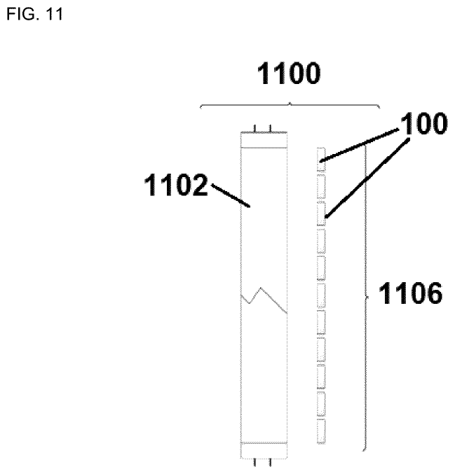

FIG. 11 depict an exemplary embodiment of an assembly 1100 including a UVC Tube type device 1102 and a plurality of UVC lens assemblies 100 arranged in a linear array 1106. The array 1106 increases UVC energy at a specific focal point. The array 1106 is mounted on the outside housing of the UVC Tube device with an insulating spacer, such as an air gap, or spacing material.

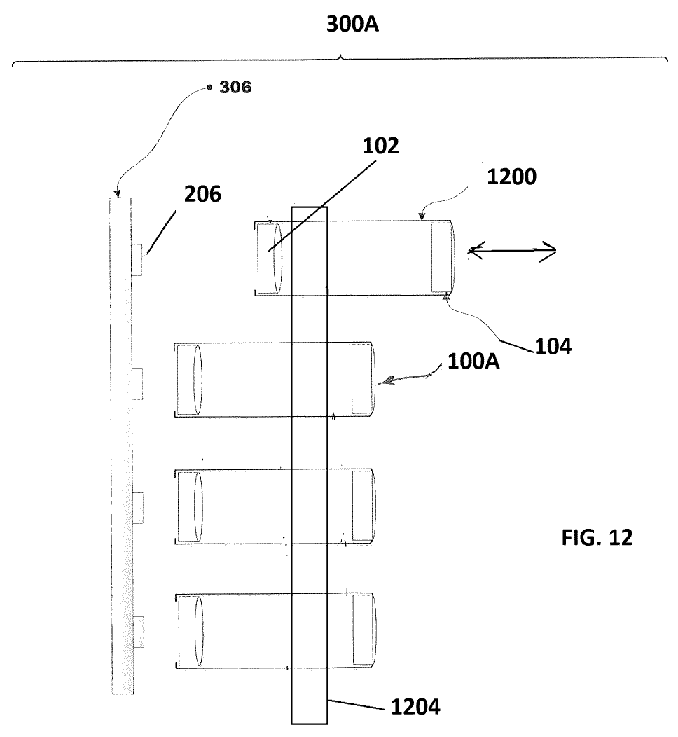

FIG. 12 illustrates an alternate embodiment, similar in some respects to FIGS. 1-3. An emitter-lens array 300A has a plurality (such as four) of UVC lens assemblies 100A. The lens assemblies 100A are in an assembly and are not physically attached to the UVC LED emitter circuit board assemblies 206 mounted on the circuit board or heat sink 306. The lens assemblies 100A are separated from the UVC LED emitter circuit board assemblies 206. The lens assemblies 100A can be held together in a frame 1204 that is molded or extruded and then placed in a position with the assemblies 100A spaced from the UVC LED emitter circuit board assemblies 206. The frame 1204 with lens assemblies 100A can be in the form of a lens cover or cap. The frame can be composed of plastic or metal. The lens assemblies can be attached to, or held by, the frame by friction, adhesive, molding with the frame, or other known method.

The assemblies 100A within a frame could be configured into a replacement cover to adapt to an existing product. The existing cover could be removed and replaced with a new cover that has the lens assemblies held in a frame.

Each assembly 100A includes a DOE lens 102 and a relay lens 104 mounted on opposite ends within a tubular housing 1200. The tubular housing 1200 is substantially hollow and acts as a spacer to set a desired distance between the two lenses 102, 104. The tubular housing can be a round cylinder, a square cross section tube, or the like.

The tubular housings 1200 can be molded with the frame 1204, adhesively secured to the frame 1204 or otherwise held by the frame 1204.

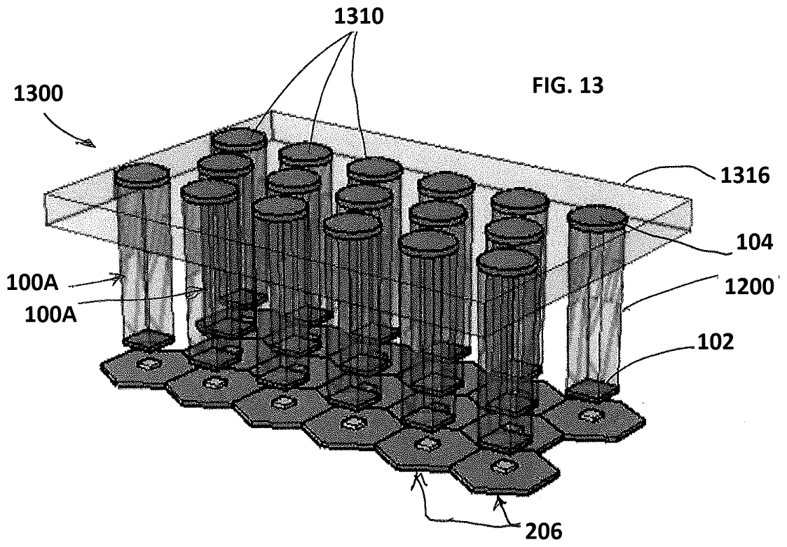

FIG. 13 depicts an alternate assembly 1300. The assembly 1300 includes a plurality of lens assemblies 100A fixed to a frame or cover 1316. Each lens assembly 100A includes a DOE lens 102 and a relay lens 104 mounted to or within a tube spacer 1200. The lens assemblies 100A, such as the quantity seventeen shown, are held together by the frame 1316 and can be placed over an array of UVC LED emitter circuit board assemblies 206 such as arranged in a honeycomb pattern (such as shown in FIG. 5). The tubular housings 1200 can be frictionally held in the frame 1316, molded with the frame 1316, adhesively secured to the frame 1316 or otherwise held by the frame 1316.

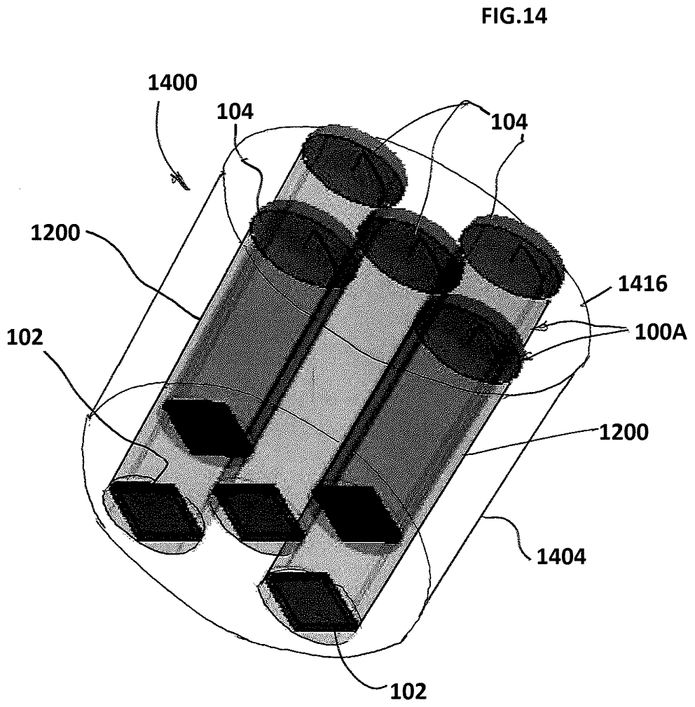

FIG. 14 depicts an alternate assembly 1400. The assembly 1400 includes a plurality of lens assemblies 100A fixed to a cylindrical frame or cover 1416. Each lens assembly 100A includes a DOE lens 102 and a relay lens 104 mounted to or within a tube spacer 1200. The lens assemblies 100A, such as the quantity seventeen shown, are held together by the frame 1416 and can be placed over an array of UVC LED emitter circuit board assemblies 206 (not shown) such as shown in FIG. 13. The tubular housings 1200 can be frictionally held in the frame 1416, molded with the frame 1416, adhesively secured to the frame 1416 or otherwise held by the frame 1416.

In the following detailed description, the UVC Beam sterilizer is described. An embodiment 1500 can be particularly useful as a water sterilizer, although not limited to such use.

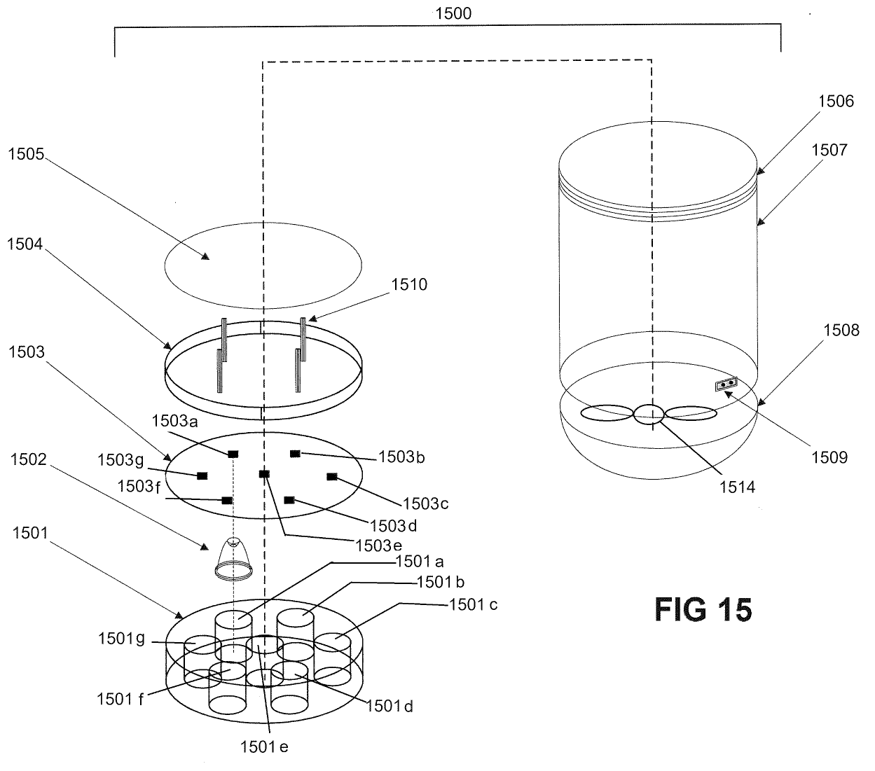

The embodiment 1500 of the UVC Beam sterilizer is illustrated in FIG. 15. The exploded perspective view of FIG. 15 illustrates an assembly that includes a lens holder 1501, a UVC Beam total internal reflection (TIR) lens 1502, an LED circuit board 1503, a heat sink 1504, a UVC LED driver/controller circuit board 1505 and mounting stand-offs 1510. This assembly of components is mounted into a sleeve 1507 which has a threaded section 1506 for mounting to piping, when used in water sterilization, or other external structure, a power input 1509 and an endcap 1508 that has a fan 1514 (shown schematically) for cooling. The sleeve is shown inverted, and during assembly would be inverted and placed over the components 1505, 1505, 1503, 1502, and 1501 with the end cap 1508 closest to the UVC LED driver/controller circuit board 1505 and the threaded section 1506 closest to the lens holder 1501.

The lens holder shown has seven cavities 1501a, 1501b, 1501c, 1501d, 1501e 1501f and 1501g for holding seven lenses 1502 (only one shown), one lens per cavity. Although seven cavities and seven corresponding lenses are described, the invention encompasses any number of cavities and lenses. The LED circuit board includes seven LED UVC emitters 1503a, 1503b, 1503c, 1503d, 1503e, 1503fand 1503g. Although seven LED UVC emitters are shown, the invention encompasses any number of LED UVC emitters. The emitters 1503a, 1503b, 1503c, 1503d, 1503e, 1503f and 1503g correspond in number and position to be in registry with the respective seven lenses 1502.

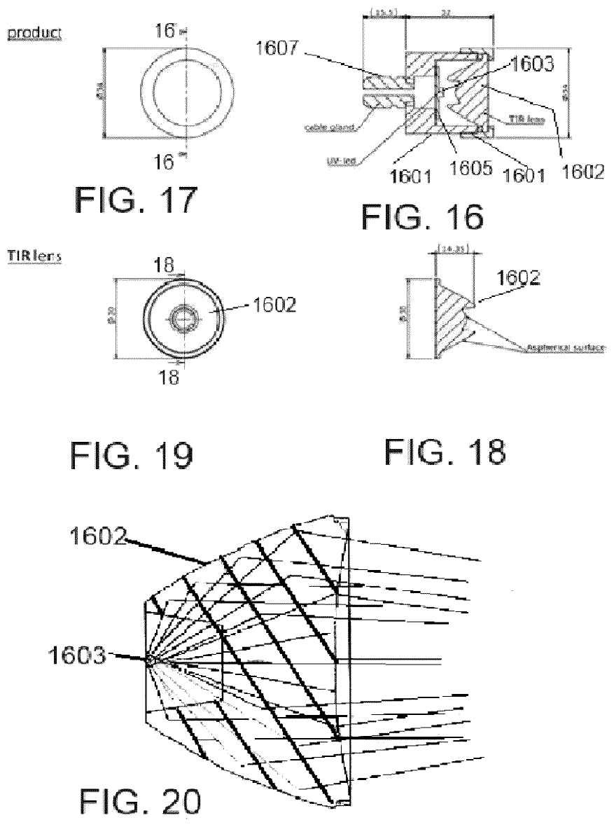

FIGS. 16-19 display an alternate embodiment assembly 1600 including lens holder 1601, UVC LED 1603 on an LED circuit board 1605 and a TIR UVC lens 1602. A cable gland 1607 allows for wires to pass into the holder 1601 This assembly shows only a single LED and lens unit compared to the seven LED and lens shown in FIG. 15.

These embodiments 1500 or 1600 can replace the assembly 200 (FIGS. 2-10) or the assembly 1200/104/102/206 (FIGS. 12-14) described in the previous embodiments, and in the various configurations described above in the previous embodiments. The DOE and relay lenses in those embodiments would be replaced by the UVC Beam TIR lens 1502 or 1602.

FIG. 20 is a schematic sectional view of a TIR lens showing the projected light beams out of the lens.

As an enhancement to the above-described embodiments, the TIR lens 1502 or 1602 can be composed of an ultraviolet-visible region transmitting glass, as disclosed in JP5904864B2. This glass is effective for UVC transmission. This glass, based on 100 wt. % of the total glass, includes:
SiO₂: 20 to 70% by weight,
Al₂O₃: 2 to 30% by weight,
B₂O₃: 15 to 40% by weight,
R¹₂O (where R¹ is Li, Na, K, Rb or Cs): 5 to 20% by weight,
R²O (where R² is Mg, Ca, Sr, Ba or Zn)): 0 to 15% by weight, and
As₂O₅ and/or Sb₂O₃: 0 to 1% by weight.

The ratio of the total number of moles of B₂O₃ and Al₂O₃ and the total mole number of the R¹₂O to the R²O is 2.5:1 to 3.5:1, and the ratio of the mole number of the Al₂O₃ to the total number of moles of R¹₂O and R²O is 1:1.

An additional component can be added selected from at least one of Fe₂O₃. WO₃, PbO, CeO₂ and TiO₂ with a total content of preferably 50 ppm by weight or less

SiO₂ is a component constituting a glass network. It is also a component that improves chemical durability. The content of SiO₂ should be 20 to 70% by weight, preferably 30 to 60% by weight.

Al₂O₃ is a component that suppresses the devitrification of the glass and enhances the chemical durability. The content of Al₂O₃ should be 2 to 30% by weight, preferably 5 to 30% by weight.

B₂O₃ is a main component of the glass network. Moreover, it is a component which improves the meltability of glass. The content of B₂O₃ should be 15 to 40% by weight, preferably 20 to 40% by weight.

R¹₂O (wherein R¹ is Li, Na, K, Rb or CS) is a component that improves the meltability of the glass. Moreover, these components can be used 1 type or 2 types or more. The total content of these should be 5 to 20% by weight, preferably 7 to 20% by weight.

R²O (wherein R² is Mg, Ca, Sr, Ba or Zn) is a component that improves the chemical durability of the glass. These components can be used alone or in combination of two or more. The total content of these should be 0 to 15% by weight, preferably 0 to 10% by weight.

As₂O₅ and/or Sb₂O₃ acts as a fining agent for the glass. These components can be used alone or in combination of two or more. The total content of these should be 0 to 1% by weight, preferably 0 to 0.5% by weight.

From the foregoing, it will be observed that numerous variations and modifications may be affected without departing from the spirit and scope of the invention. It is to be understood that no limitation with respect to the specific apparatus illustrated herein is intended or should be inferred.

The invention claimed is:

1. An array comprising plural ultraviolet C (UVC) light sources which focus UVC light energy from a UVC light source into a UVC beam to fucus UVC light energy at a set distance.

2. The array according to claim 1, wherein each UVC light source comprises a UVC beam total internal reflection (TIR) lens, wherein the TIR lens is composed of:
SiO₂,
Al₂O₃,
B₂O₃,
R¹₂O (where R¹ is Li, Na, K, Rb or Cs),
R²O (where R² is Mg, Ca, Sr, Ba or Zn), and
As₂O₅ and/or Sb₂O₃.

3. The array according to claim 1, wherein each UVC light source comprises a UVC beam total internal reflection (TIR) lens, wherein the TIR lens is composed of:
SiO₂ at 20 to 70% by weight,
Al₂O₃ at 2 to 30% by weight,
B₂ O₃ at 15 to 40% by weight,
R¹₂O (where R¹ is Li, Na, K, Rb or CS) at 5 to 20% by weight,
R²O (where R² is Mg, Ca, Sr, Ba or Zn) at 0 to 15% by weight, and
As₂ O₅ and/or Sb₂ O₃ at 0 to 1% by weight.

4. The array according to claim 3, wherein the plural UVC light sources are arranged along an arc.

5. The array according to claim 3, wherein the plural UVC light sources are arranged along a linear pattern.

6. The array according to claim 3, wherein the plural UVC light sources each have a polygonal perimeter and are arranged in a honeycomb pattern.

7. The array according to claim 3, arranged forming a four-sided UVC sterilization device with an interior volume.

8. The array according to claim 3, arranged forming a UVC sterilization chamber with an interior volume.

9. The array according to claim 3, wherein the array is enclosed within a UVC Fiber Optics Amplifier device, wherein UVC energy is focused to a single point leaving the UVC Fiber Optics Amplifier device.

10. The array according to claim 1, wherein each UVC light source comprises a Diffractive Optical Element with a beam width of UVC energy, which transmits a UVC beam through a Relay Lens which thereby allows an extended distance of UVC energy.

11. The array according to claim 10, wherein the Diffractive Optical Element comprises a series of spacers and lenses that allow the manipulation of a wide angle UVC light source for the purpose of focusing the light to a desired beam shape.

12. The array according to claim 1, wherein the plural UVC light sources are arranged along an arc.

13. The array according to claim 1, wherein the plural UVC light sources are arranged along a linear pattern.

14. The array according to claim 1, wherein the plural UVC light sources each have a polygonal perimeter and are arranged in a honeycomb pattern.

15. The array according to claim 1, wherein each UVC light source includes a lens assembly including:
a first spacer;
a DOE lens;
a second spacer; and
a relay lens.

16. The array according to claim 15, wherein the relay lens includes a mount for the lens assembly to attach to a UVC LED board.

17. The array according to claim 1, arranged forming a four-sided UVC sterilization device with an interior volume.

18. The array according to claim 1, arranged forming a UVC sterilization chamber with an interior volume.

19. The array according to claim 1, wherein the array is enclosed within a UVC Fiber Optics Amplifier device, wherein UVC energy is focused to a single point leaving the UVC Fiber Optics Amplifier device.

20. An array comprising a UVC light source and a plurality of lens assemblies, each having a Diffractive Optical Element with a beam width of UVC energy, which transmits a UVC beam through a Relay Lens which thereby allows an extended distance of UVC energy. which focus UVC light energy from a UVC light source into a UVC beam to fucus UVC light energy at a set distance.
